(19) United States
(12) Patent Application Publication
(10) Pub. No.: US 2014/0073879 A1
(43) Pub. Date: Mar. 13, 2014

(54) SYSTEM FOR MONITORING PREGNANCY IN MAMMALS

(71) Applicants: Divya Cantor, Louisville, KY (US); Barry Simon, Lexington, KY (US); Vasily Abramov, Louisville, KY (US); David Phelps, Louisville, KY (US); John E. Vanderburgh, Louisville, KY (US); Joshua White, Louisville, KY (US)

(72) Inventors: Divya Cantor, Louisville, KY (US); Barry Simon, Lexington, KY (US); Vasily Abramov, Louisville, KY (US); David Phelps, Louisville, KY (US); John E. Vanderburgh, Louisville, KY (US); Joshua White, Louisville, KY (US)

(73) Assignee: MD DEVICE LLC, Louisville, KY (US)

(21) Appl. No.: 13/611,265

(22) Filed: Sep. 12, 2012

(51) Int. Cl. A61B 5/00; A61F 6/08; A61B 5/01; A61B 5/053; A61B 5/145

(52) U.S. Cl. CPC A61B 5/435; A61B 5/0011; A61B 5/6875; A61B 5/053; A61B 5/14539; A61B 5/01; A61F 6/08; A61B 5/0022; A61B 5/746
USPC 600/301; 600/304

(57) ABSTRACT

A system is disclosed for long term, continuous, monitoring of pregnant mammals particularly to detect the onset of preterm labor. For example, the system enables communication between sensors and a collection unit for transmittal to a remote unit for display and monitoring of the collected data by a clinician while the individual is ambulatory and not in the presence of the clinician.

### FIELD OF THE INVENTION

This invention relates to medical monitoring in general and more particularly to a system for monitoring pregnancy.

### BACKGROUND OF THE INVENTION

The birthing process in mammals has a normal or average duration, for example in humans gestation is normally 266 days while for horses it normally ranges between 335 and 345 days. For cattle gestation averages 283 days while for swine about 113 to 116 days and for sheep 144 to 151 days. The actual period for human gestation is variable but is considered full-term to conclude between 38-42 weeks post-conception. In humans, for example, labor and birth that commences earlier than 36 weeks as a result of a high risk pregnancy or other complications presents serious if not fatal problems for the newborn infant that can well continue on to adulthood. Delivery at 30-32 weeks normally necessitates considerable time in an NICU and delivery very early at around 23 weeks is considered almost uniformly fatal

For most domestic animals entering into labor and the birthing process occurs with little assistance. However, for humans in particular and certain valuable domestic animals, such as Thoroughbred horses, the gestation period in the final trimester is normally closely monitored for the beginning of labor and for complications such as preterm labor resulting in preterm birth.

For domestic animals a preterm birth will usually produce a still born or a weak and sickly animal requiring expensive attention. Especially in the case of Thoroughbred horses where stud fees may run up to six figures, a premature foal may not survive or perform to the expectations of the breeder resulting in a substantial financial loss.

For humans the societal burden of preterm births in the United States was estimated to be $26 billion in 2005 by the Committee on Understanding Premature Birth and Assuring Healthy Outcomes under the auspices of the US Institute of Medicine. These costs include medical care services at $16.9 billion; maternal delivery costs $1.9 billion; early intervention services $611 million; special education services associated with a high incidence of disabling conditions associated with premature infants added $1.1 billion. Finally, the committee estimated that lost household and labor market productivity associated with those disabling conditions contributed $5.7 billion.

High-risk pregnancies are not always detected early nor easily determined in advance so that preventative measures can be taken to avoid a preterm birth. Women pregnant for the first time or who may not have access to medical help can present a high-risk pregnancy without being aware of their condition. Early identification of high-risk pregnancies facilitates monitoring and prompt initiation of therapy. Early initiation of therapy has the potential to decrease the onset of labor, thus reducing the complications endured by the newborn as well as reducing the cost burden. However, monitoring of even normal pregnancies in the last trimester is also recommended if only to avoid unnecessary trips to the hospital because of false labor.

It is recommended that monitoring of a pregnancy, especially in the last half of the pregnancy, be conducted frequently so that complications can be identified as well as the onset of labor in its very early stages. In the case of domestic animals monitoring of the animal's pregnancy will normally require the on-site presence of a veterinarian which may not be convenient.

In the United States and other Western countries where medical services are more readily available, a pregnancy may be monitored at the doctor's office as an outpatient or in the hospital. However, this requires a visit to the doctor's office or the hospital every few days in order to monitor the patient's progress. In developing countries it may not be possible for a woman to see a doctor on a regular basis so that intervention to alleviate or treat a condition may be too late.

Various systems have been put forward to monitor the status of a pregnancy. For example U.S. Pat. No. 6,270,458 describes a system employing ultrasound to monitor the dilation of the cervix and descent of the presenting part during labor. This system employs small ultrasound reflectors disposed on either side of the cervical os and on the fetal presenting part to reflect ultrasound signals transmitted by an ultrasound interface unit located outside of the body.

Measurement of uterine electrical activity, electrohysterography, has been proposed as an alternative approach to the detection of labor. For example WO 94114373 (Garfield) records signals from embedded electrodes, WO 95/31932 and WO 96/39931 (Garfield) disclose a method which stores data and compares activity and U.S. Pat. No. 5,373,852 (Harrison) uses radiotelemetric transmission for sensing pressure, temperature and electrical activity. Rosenberg (WO 97/25922) discloses a further method for analysis of electromyographic data. These techniques relate exclusively to clinical settings, usually intrusive systems for data collection. U.S. Pat. No. 6,823,211 discloses a nonintrusive_device including recording electrodes, a digital converter and a display unit that can be_worn on the exterior of the abdomen or vaginal area of a body for the analysis of uterine electrical activity to monitor the progress of, and/or diagnose active labor and can be used to obtain an indication of uterine preparedness for labor in the initial phase of parturition before onset of active labor. Another system that relies on the measurement of uterine electrical activity is disclosed in U.S. Pat. No. 6,290,657 that, similar to the '211 patent, uses surface electrodes and a monitor to sense and analyze electrical signals that are transmitted by telephone to the physician's office.

The human birthing process is divided into four stages. Stage 1 occurs from the onset of labor to full cervical dilation. Stage 2 is from full cervical dilation to delivery; stage 3 from delivery to expulsion of placenta and stage 4 from expulsion of the placenta to afterbirth recovery. Thus, monitoring the physiological parameters of the cervix in humans and mammals allows the clinician to follow the progress of a pregnancy during stage 1 of the process where complications, particularly preterm labor, can be recognized and possibly treated. In that connection some successful work has been done in this area but the methods employed require the presence of the clinician and usually employ invasive procedures.

For example, in Patent Application Publication U.S. 2009/0137925 the pregnancy is monitored by attaching a clip carrying a pair of tetra-pole electrodes to the patient's cervix. The electrodes that are electrically connected to a transducer cable that supplies the signal to an interface unit and monitor. The transducer cable carries analog to digital conditioning hardware while the interface unit includes an algorithm for comparing typical impedance value with the impedance value of the cervix being measured. The resulting output can be a measure of time of labor vs. cervical stromal impedance (CSI) value or time in labor vs. cervical dilation and/or effacement for plotting a Friedman labor curve.

The prior art methods are inconvenient and are not suited for long term, continuous monitoring of the patient. In many cases monitoring involves the intervention of a clinician that requires the patient to visit the hospital or the clinician's office frequently to undergo the monitoring procedures. Placing sensors externally may introduce discrepancies in the data collected. For example, discrepancies due to variations in skin temperature and variations in contact pressure between the skin and the sensor. Other methods require that relatively large devices be placed in the patient that requires frequent removal to avoid odor and possible infection. Several methods require connection by wire to sensors at one end and to burdensome equipment at the output end that requires that the patient be non-ambulatory. In addition, many prior art systems are_concerned only with the measurement of vaginal contractions which can be an unreliable indicator of the onset of labor.

### SUMMARY OF THE INVENTION

The physiological parameters of cervix effacement (length) and ripeness (thickness) and dilation are reliable indicators of the onset of labor. Data based on these parameters can be plotted to produce a curve having a slope that normally is relatively flat but which undergoes a significant change due to physiological changes of the cervix as labor becomes imminent. Such a curve can be used to determine if an individual is approaching actual labor as contrasted with false labor and is a reliable indicator of preterm labor. In addition other physiological parameters indicative of cervix condition and female health are pH and temperature.

The present invention relates to a system for long term, continuous, monitoring of pregnant mammals particularly to detect the onset of preterm labor. For example, the system enables communication between sensors and a collection unit for transmittal to a remote unit for display and monitoring of the collected data by a clinician. In accordance with the invention after an initial examination it is not ordinarily necessary for the individual being monitored to be in the presence of the clinician.

Accordingly, it is an object of the invention to monitor and record the physiological condition of the cervix from sensors immediately adjacent to and in contact with the cervix.

An object of the invention is to provide a system for continuous monitoring of the condition of the cervix while permitting the subject to be completely ambulatory and have complete freedom of action.

Another object of the invention is to provide a system wherein the monitoring of the cervix can be done remotely.

A further object of the invention is to continuously monitor changes in effacement, dilation and ripeness of the cervix to monitor the condition and/or the stage of pregnancy.

A further object of the invention is to continuously monitor pH and temperature as physiological parameters of the cervix and the genital health of the individual.

Another object of the invention is to provide a system that can be employed early in pregnancy to detect the early onset of conditions that indicate the likelihood of a preterm birth.

It is an object of the invention to provide a system m which data is wirelessly communicated to and recorded in an interface device”.

It is an object of the invention to provide a system that collects and analyses data and distinguishes between normal conditions of the cervix and relevant changes in effacement and ripeness that may indicate early stages of labor.

According to one aspect of the invention there is provided a method for monitoring the birth process of a mammal comprising the steps of:
a. determining the condition of the cervix as it relates to the onset of labor and birth by measuring physiological parameters of the cervix as a determination of effacement and ripeness;
b. conducting signals generated by the measurement of the electrical parameters to the interface device;
c. processing the data to create a baseline of cervical condition;
d. continuously transmitting signals and collecting and processing data therefrom to determine cervical changes in relation to the baseline;
e. wirelessly transmitting the collected data from the interface device to an interface unit; and
f. transmitting the data from the interface unit to a remote unit for display and monitoring by the clinician.

In accordance with the method of the invention the data is developed and can be remotely monitored by a data collection center or by a clinician while the individual is completely unrestrained and ambulatory. Thus the need for frequent contacts with a clinician such as a doctor or veterinarian is unnecessary since the the condition of the cervix can be remotely monitored at the data collection center, the clinician's office or hospital.

The physiological parameters of the cervix including impedance, body temperature and pH can be measured by several relatively noninvasive technologies which permit the development of data while permitting the individual to be completely free to move around. For example, cervix effacement, dilation and ripeness can be measured using ultrasound, infrared or by measurement of impedance.

In one preferred mode the data includes impedance measurements and is conveniently processed to correlate to cervical stromal impedance (CSI) values stored in the database of the interface device. Typical values may be found in Gandhi, et al., “Comparison of Human Uterine Cervical Electrical Impedance Measurements Derived Using Two Tetrapolar Probes of Different Sizes,” European Journal of Obstetrics & Gynecology and Reproductive Biology, Vol. 129, Issue 2, December 2006, 145-149, which is incorporated herein by reference in its entirety.

Alternatively, in another embodiment the processed data is accumulated to generate baseline data for the individual being monitored. The baseline data can be displayed as a curve or displayed in textual form. In either case a significant change in the slope of the curve or an increase or decrease in data values is an indication that a significant change in the physiological condition of the cervix and that labor may be imminent. In the case of pH and temperature measurements the data is accumulated to generate a baseline of these parameters for the individual being monitored.

In another aspect of the invention the system comprises:
a. at least one sensing device to detect the physiological parameter being measured and for generating a digital signal in response thereto;
b. an interface/telemetry device for receiving, storing and analyzing the signals generated by the sensing device to develop data relating to the physiological parameter being measured and for wirelessly transmitting the signals to a transmitting unit;
c. the transmitting unit for wirelessly transmitting the data received from the interface/telemetry device to a remote location; and
d. a remote unit for receiving and displaying the data.

Depending on the technology employed to monitor the physiological parameters of the cervix the sensor may take several well understood forms. Thus, ultrasound and electrical measurements have been employed to measure contractions of the uterus to predict the onset of labor. These same techniques can be employed to monitor physiological parameters of the cervix. With ultrasound, for example, reflectors may be placed on either side of the cervical os that reflect ultrasound signals from an interface unit located outside of the body. Changes in physiological parameters of the cervix such as effacement, dilation or ripeness will produce changes in the reflected signals. In the case of electrical measurements, such as impedance, electrodes are positioned immediately adjacent to the cervix and the change in the output signal correlated with effacement, dilation and ripeness of the cervix. Likewise the sensing device can include one or more pH microelectrodes and temperature sensing electrodes. Temperature and pH are indicators of other systemic or gynecological problems that can be monitored to suggest that medical treatment is required.

In accordance with the invention these techniques can be employed early on in the pregnancy with no discomfort to the individual and without the necessity of restraining or otherwise preventing the free movement of the individual during the monitoring process.

To maintain freedom of movement for the individual being monitored all transmissions of data, i.e. from the sensor to the interface device which is external of the body and thence to the transmission unit is wireless. Since the transmission distance between the interface device and the transmission unit will be relatively short the choice of transmission means is not critical. Thus, blue tooth technology is applicable as it is easily adapted for the purpose and has low power requirements. For transmission from the interface unit to the remote unit a more robust system is required since the transmission distance can be long, on the order of a few miles to many miles depending on the location of the remote unit. Accordingly, transmission may be by telephone, landline or cellular, or by a radio frequency link (RF).

These and other objects and advantages will become apparent from the following description of the invention taken in conjunction with the drawings in which:

### BRIEF DESCRIPTION OF THE DRAWINGS

FIG. 1 is a block diagram of the components of an apparatus in accordance with the invention;

FIG. 2 is an isometric view of one embodiment of a sensing device in accordance with the invention;

FIG. 3 is a cross sectional view of the sensing device of FIG. 2 positioned adjacent the cervix;

FIG. 4 is an isometric view of an embodiment of the sensing device that functions as a pessary;

FIG. 5 is a cross sectional view of the sensing device of FIG. 4 positioned adjacent the cervix;

FIG. 6 is an isometric view of an embodiment of the sensing device adapted for suturing onto the cervix and illustrating the power supply extending from the body of the sensing device;

FIG. 7 is a cross sectional view of the sensing device of FIG. 6 positioned adjacent the cervix;

FIG. 8 is an isometric view of the sensing device consisting of a flexible ring defining open ends and having a power supply and electronics compartment extending from one of the open ends;

FIG. 9 is a sectional view showing the device of FIG. 8 positioned around the cervix;

FIG. 10 is an isometric view of an embodiment of the invention in which the sensing device also functions as a constricting pessary;

FIG. 11 is an isometric view of a sensing device consisting of an outer ring and inner coaxial ring inter connected by a membrane;

FIG. 12 is an embodiment of a sensing device adapted to rest against one side of the cervix and the adjacent vaginal wall;

FIG. 13 is another embodiment of a sensing device similar to FIG. 12 but having an extending compartment for the battery and electronics; and

FIG. 14 is a flow diagram illustrating the steps in the operation of the system of the invention.

### DESCRIPTION OF THE INVENTION

The system of the invention is suited for monitoring the pregnancy of mammals, however for ease of description the invention will be described in connection with monitoring the pregnancy of a human female. Although other systems employing infrared and ultrasound are within the scope of the invention, the preferred system described herein will be in connection with the utilization of impedance as an indicator of the physiological condition of the cervix.

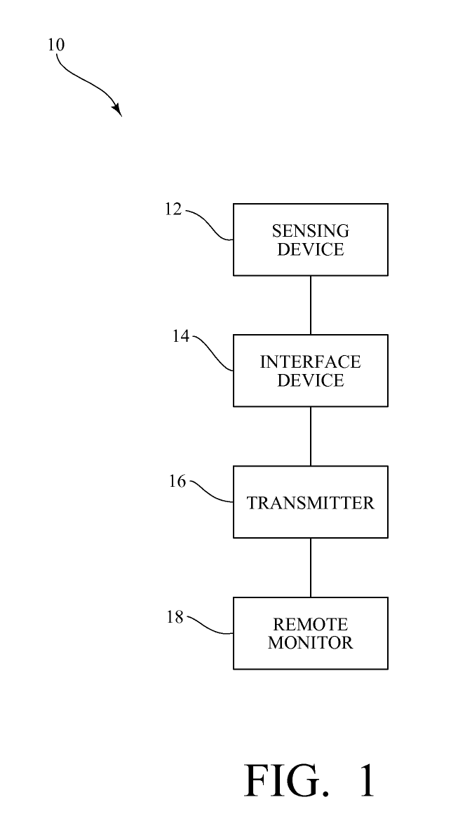

Referring to FIG. 1 one embodiment the system of the invention, shown generally as 10, is designed for the measurement of the physiological parameters of impedance, pH and temperature as indicative of the physiological condition of a cervix and health of the individual. The system 10 is schematically illustrated as comprising a sensing device 12 that is designed for the non-invasive placement in the body for the detection of the physiological parameters. The sensing device 12 includes sensing electrodes for the measurement of impedance, temperature and pH and further contains electronics for conversion of the measured signals to digital data.

The sensing device 12 wirelessly communicates with an interface device 14 that receives the digital data from the sensing device. The interface device 14 will normally be worn by the individual outside of the body, such as by attachment to the individual's clothing or will otherwise be in close proximity to the individual's body.

The interface/telemetry device 14 includes a housing that contains electronics having software which is programed to compare the data as received to a data base consisting of values for the physiological parameters being monitored. The interface device 14 further includes memory containing the data base of values for the parameters or, as is often the case where typical values are not available, to accumulate the data as they are received to create a data base of parameters for the individual against which newly received data are compared. The software includes the corresponding algorithms for creating impedance, pH and temperature baseline data and for comparing the data of each parameter to the baseline data for the respective parameter. A significant change in received data from the baseline data will cause the software program to transmit a warning to the clinician that some action is required.

At predetermined time intervals the analyzed data that are received during a preceding interval may be transmitted to a transmitter 16 for transmission to a remote monitor 18 for read out of the analyzed data. The transmitter 16 may be any device capable of receiving the signal from the interface device 14 and transmitting it over relatively long distance. It may be a separate unit or may be integral with the interface device 14. Thus, good results are obtained when transmission is, for example, a telephone, either land line or cellular, or a radio receiver/transmitter. When the transmitter 16 is separate from the interface device 14 communication between the interface device 14 and the transmitter 16 preferably is wireless or, if the interface device is not being worn by the individual, the devices may be hardwired.

Because the sensing device 12 and the interface device 14 are normally in relatively close proximity communication between the sensing device 12 and the interface device 14 is most conveniently achieved using Zigbee, wifi, radio frequency or Bluetooth®.

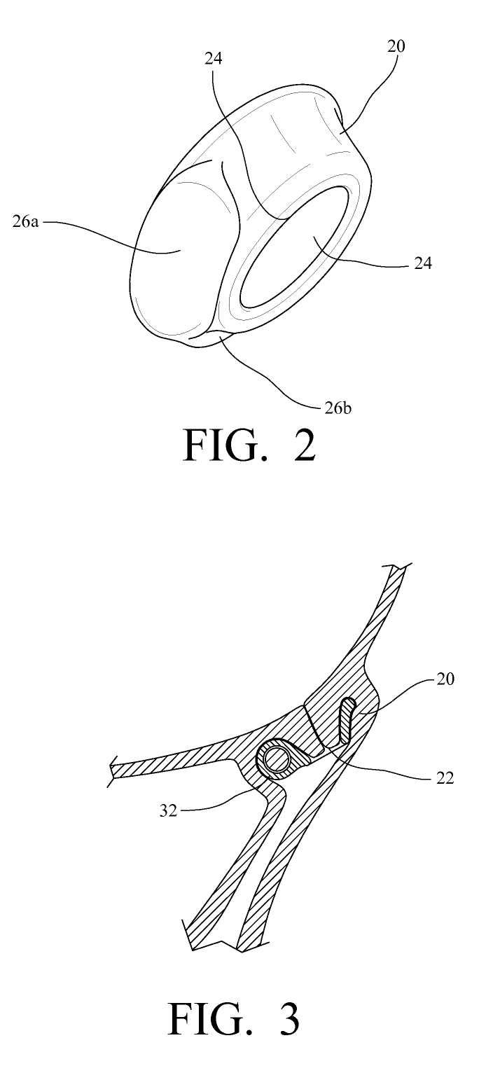

As illustrated in FIG. 2 and FIG. 3, in one embodiment the sensing device 12 comprises a flexible annulus 20 disposed around a cervix 22 and is contiguous therewith. The annulus 20 is formed of a resilient, non-conductive, biocompatible material, such as polyurethane, silicone or silicone rubber that exhibits the desired properties of resiliency, flexibility, biocompatibility and non-conductivity. The inner radial surface of the annulus 20 is provided with one or more electrodes 24 that are in electrical communication with signal converting electronics for converting raw impedance measurements to digital data. The annulus 20 being flexible and resilient can be slightly stretched between the fingers of a clinician for placement around the cervix 22. When the clinician's fingers are removed the annulus 20 will return to its original diameter to provide intimate contact between the inner radial surface, the electrodes 24 and the cervix 22. The intimate contact serves to retain the annulus 20 in position on the cervix and to insure intimate contact between the cervix 22 and the electrodes 24.

As more clearly shown in FIG. 3 a pair of opposed enlarged tubular members 26a and 26b are disposed on the annulus 20. The members 26 a and b are open at their facing ends 28 and closed at their opposite ends. The respective bores of the members 26a and 26b each define a compartment 32 in which are contained the battery power supply for the sensing device and the electronics for converting the raw impedance signals to digital data. The open ends 28 may be fused together during the manufacture of the sensing device 20 after the power supply 34 and electronics 36 are inserted in their respective compartments 32.

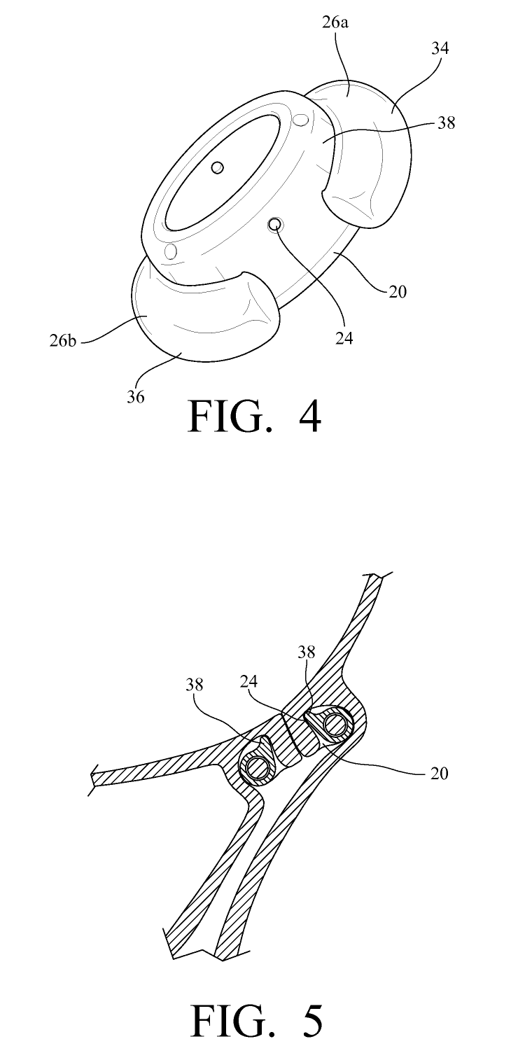

FIG. 4 illustrates another embodiment of the sensing device 12 comprising the annulus 20, electrodes 24, and compartments for the power supply 34 and electronics 36 defined by members 26 a and b as shown in FIG. 1 which are closed at both ends and disposed oppositely on the annulus. In addition, however, an extending annular skirt 38 is formed on the inner surface of the annulus 20. The sensing device functions as described above in measuring impedance. However, as shown in FIG. 5 when the annulus 20 is disposed on the cervix 22 the annular skirt 38 is urged against the cervix and may function as a pessary to provide support for the cervix and to aid in maintaining the annulus in position on the cervix.

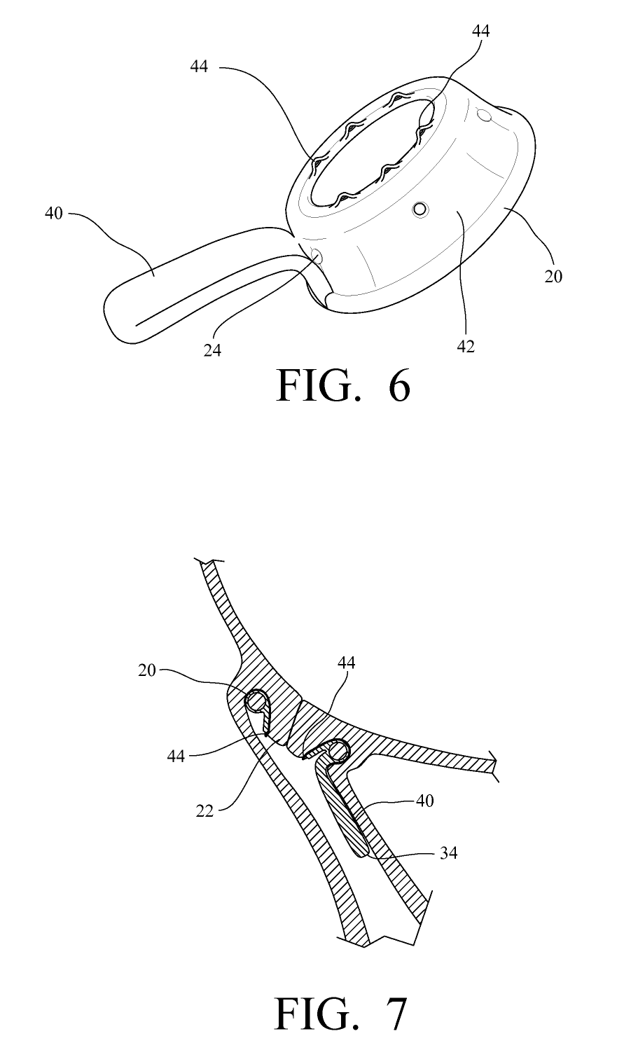

Depending on the patient, under certain circumstances the sensing device 12 may tend to shift over time after being placed around the cervix 22. This can result in inaccurate data generated by the sensing device 12. As shown in FIG. 6 and FIG. 7 this embodiment of the invention is designed to prevent shifting of the sensing device 12. the sensing device 12 comprises the annulus 20 and one tubular member 26b that defines the compartment for the electronics. An annular skirt 42 extends downwardly from the annulus 20. A plurality of eyes 44 are formed on the extending end of the skirt 42. The eyes 44 serve as suture points for suturing the sensing device 12 to the cervix 22. A separate battery container 40 extends downwardly outwardly from the annulus 20 to serve as the power supply for the electronics. The extending battery container 40 is configured to fit against the vaginal wall adjacent the cervix 22. In addition the extending battery container 40 will serve as a convenient handle for manually supporting the sensing device 12 during the suturing procedure and for placement and removal of the sensing device.

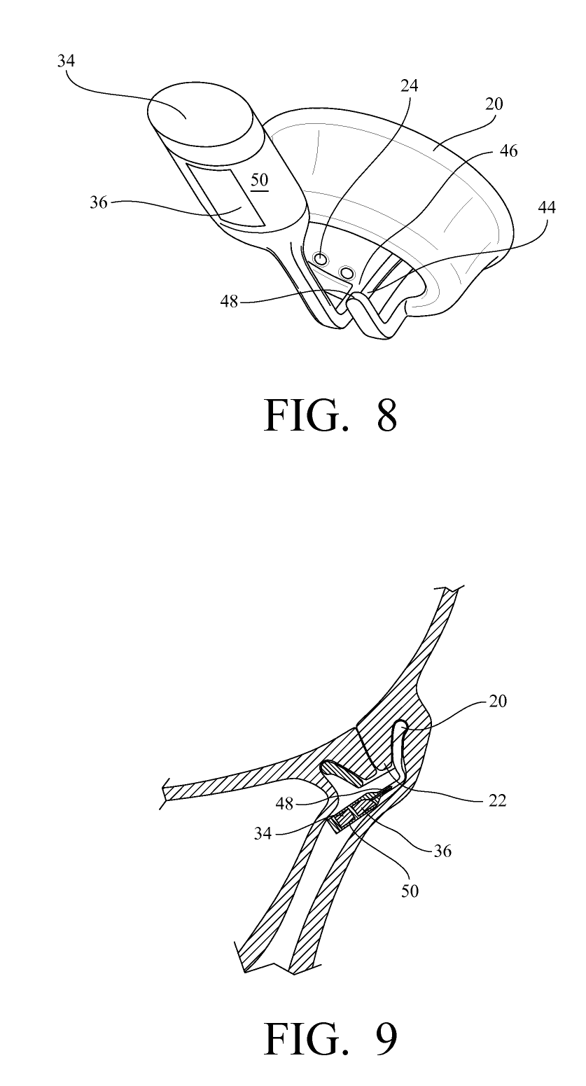

Yet another embodiment of the sensing device 12 is illustrated in FIG. 8 and FIG. 9 in which the annulus 20 is interrupted at 44 to define free ends 46 and 48. Free end 48 extends downwardly away from the annulus 20 and is enlarged to define the compartment 50 for the battery 34 and electronics 36. As shown in FIG. 9 the extending end 48 is sufficiently flexible to allow the compartment 50 to lie in the vagina below the cervix.

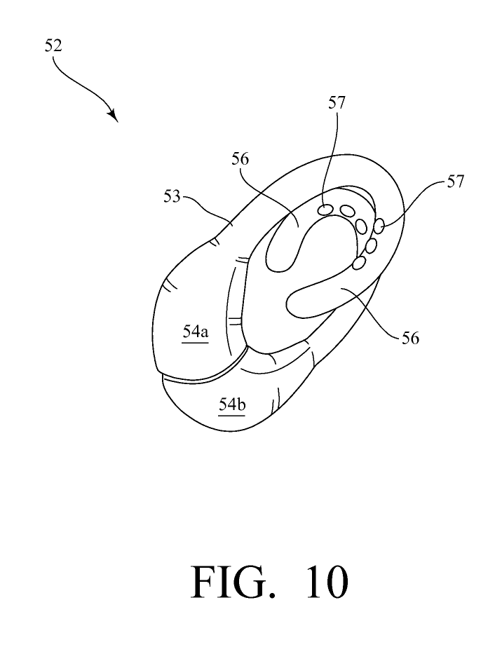

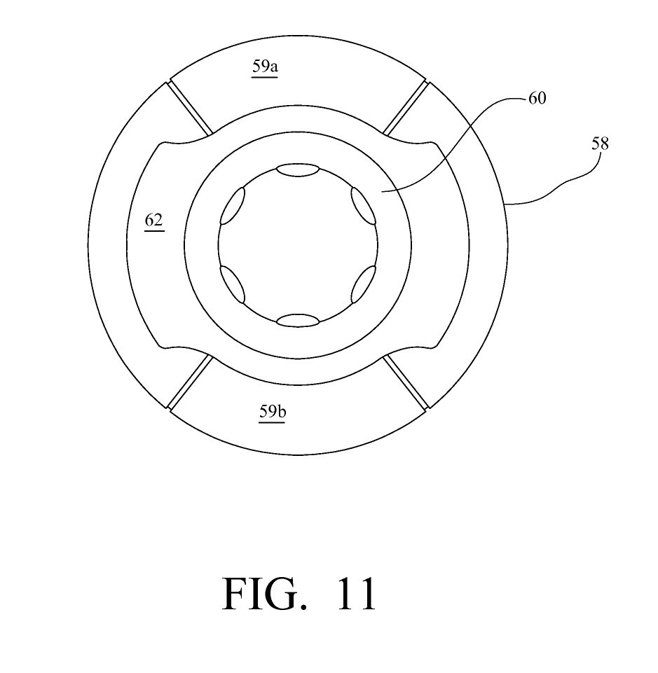

In another embodiment shown in FIG. 10 the sensing device 12 is configured as a constricting pessary to maintain closure of the cervix. In this embodiment the sensing device comprises a flexible annulus 53 on which is formed enlarged compartments 54a and 54b in which are respectively disposed the battery and the electronics The annulus 53 is interrupted to define a pair of resilient, flexible U-shaped arms 56. Electrodes 57 are disposed in the arms 56 that communicate with the electronics for measurement of the physiological parameters of the cervix and vaginal area around the cervix. The sensing device 52 is positioned around the cervix by spreading the U-shaped arms 56. Once positioned, the arms 56 are allowed to return to their original configuration so that each arm securely contacts the cervix to provide constrictive support as a pessary and to maintain the position of the sensing device 12 on the cervix.

FIG. 11 illustrates a form of sensing device 12 that comprises an outer annulus 58 and an inner concentric annulus 60 joined by a membrane 62. The battery and the electronics are in electrical communication and are contained respectively in compartments 59a and 59b formed on the outer annulus 58. Electrodes 61 are disposed on the inner concentric annulus 60 for impedance measurement. The membrane 62 is electrically conductive and the electrodes 61 communicate with the electronics through the membrane. The inner annulus 60 is placed about the cervix and the outer annulus 59 is supported by the vaginal wall to aid in maintaining the sensing device 12 in position.

Although the system has been described thus far in connection with a sensing device in the form of annulus the surrounds the cervix good results can be achieved with a sensing device that can be positioned adjacent one side of the cervix and the facing vaginal wall. Such embodiment is convenient to place and can be readily removed for cleaning and repositioned by the patient or clinician.

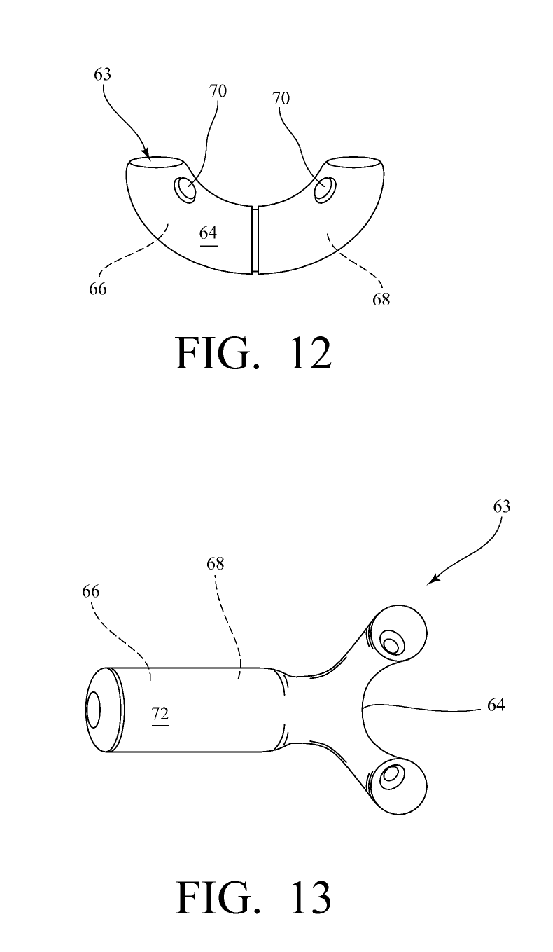

Referring to FIG. 12 there is illustrated one embodiment of a sensing device 63 which comprises an arc shaped body 64 in which are contained a battery 66 and electronics 68. Electrodes 70 in communication with the electronics 68 and battery 66 are disposed on the outer surface of the body for sensing the physiological parameters of the cervix and surrounding vaginal area. Operation of the sensing device 63 is as described for the sensing device 12 of FIG. 2.

Depending on the individual being monitored, a bladder (not shown) containing a vaginally inert fluid, such saline solution, may be positioned between the body 64 and the adjacent vaginal wall to aid in maintaining the body in close contact with the cervix.

FIG. 13 illustrates an embodiment of the sensing device of FIG. 12 but additionally includes an extending compartment 72 which contains the battery 66 and electronics 68. The extending compartment 72 projects into the vagina and rests against the vaginal wall when the sensing device 63 is in position to provide additional support for maintaining the body in proper position against the cervix. In addition the extending compartment is a convenient gripping area for inserting and removing the sensing device 63.

It will be understood that monitoring of the physiological parameters of a pregnancy requires that body temperature and pH may also be monitored and recorded. The various embodiments of the sensing devices described herein are provided with the appropriate sensors for detecting body temperature and pH. These sensing devices are well understood in the art and do not per se form a part of this invention. For example pH can be measured by ion sensitive field effect transistors (ISFET) and miniaturized electronic temperature sensors for converting pH and temperature signals to digital data and holding this data in separate packets for transmission to the interface device 14 for inclusion in a data base.

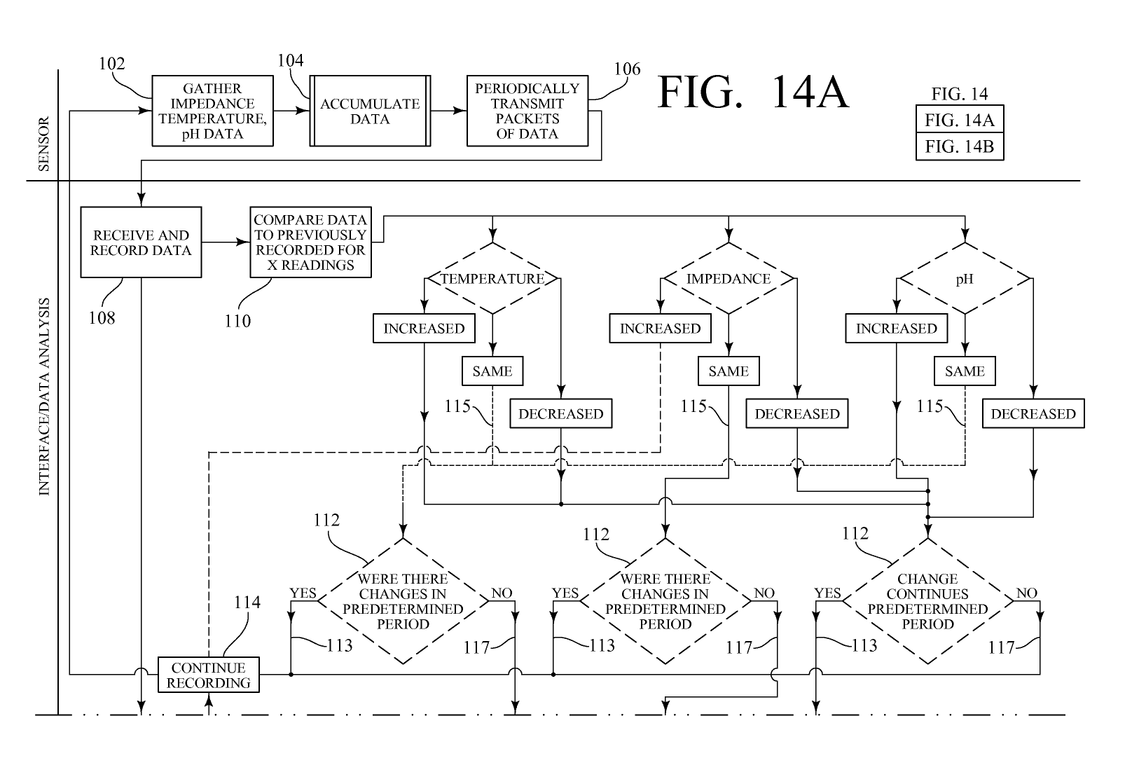

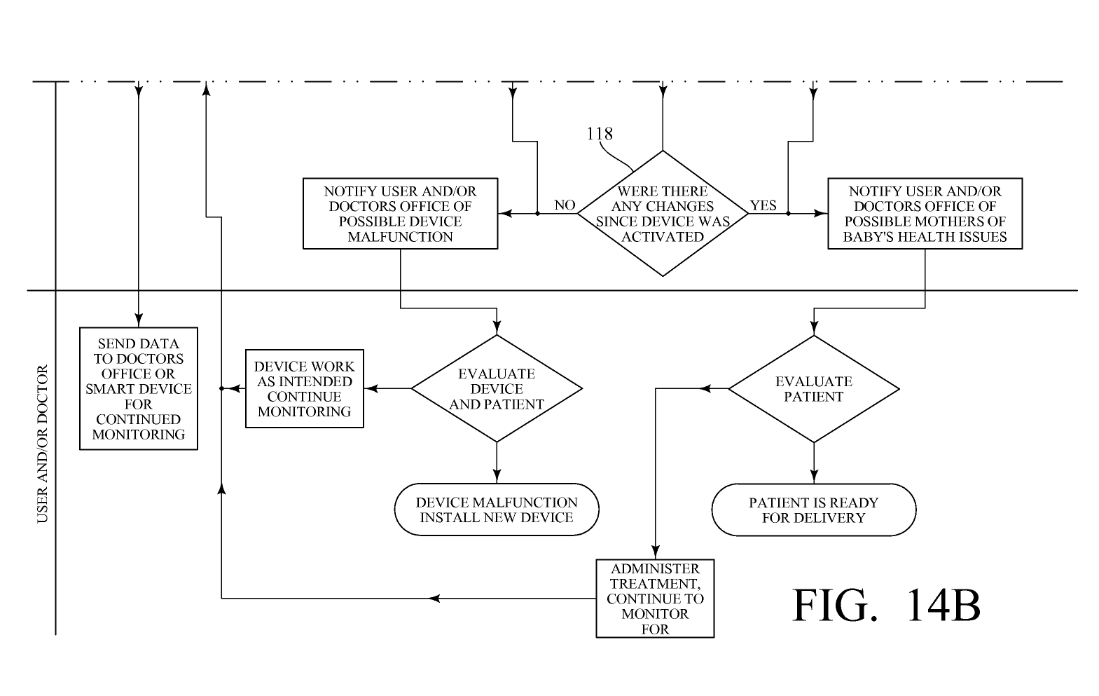

FIGS. 14A and B are an operation schematic illustrating the operation of the system and its major components, sensor 12, interface 14 and monitor 18. The sensing device 12, in contact with the cervix, in step 102 gathers impedance, pH and temperature signals and converts the signals into digital data. The data are accumulated at step 104 and periodically transmitted as a packet of each of the parameters, impedance, pH and temperature, to the interface device 14 in step 106. In step 104 the data may be accumulated at the sensor 12 over a period ranging up to several hours and transmitted as a packet less frequently to conserve battery life. The data packets are transmitted wirelessly by any convenient wireless transmission technique such as Bluetooth®, Rf or infrared. Bluetooth® is the preferred technique as being the most easily and economically incorporated in a sensing device 12 and because the sensing device normally will be in close proximity to the interface device 14 during data transmission the transmission distance is short and power requirements are low.

The interface device 14 receives and records the transmitted data in step 108 and compares the received data with previously recorded data at step 110. Data representing temperature, impedance and pH for each transmitted packet of data is analyzed by being compared either to a data base consisting of typical values or to a data base consisting of previously recorded data for the individual at step 110 to determine an increase, no change or decrease of the data values. The newly received data is stored in memory and, in the case where the data base consists of previously recorded data for the individual being monitored, is added to the data base.

After comparing the newly received packet of data with the baseline for any or all of the parameters of temperature, impedance and pH and there is no substantial change from the baseline, monitoring steps 104 through 110 will continue. In step 112 a change in the trend of the data values for any or all of the parameters of temperature, impedance and pH for each transmitted packet of data over a predetermined period, for example 8 hours, will trigger a signal 113 to the user and the clinician to more closely monitor data output or to personally examine the individual. In the meantime monitoring and recording of the parameters is continued at 114. Thus, for example, a decreasing trend of data for impedance may indicate a change in cervix effacement, ripeness or dilation indicating the onset of birth. In the case of impending preterm birth steps may be taken to delay the birthing process and thus allow for further development of the fetus. Likewise, a trend showing a change over time in pH and or temperature may indicate infection or other health issue that can be treated by the clinician.

In the event there are no substantial changes from the recorded data of a received data packet for a parameter, signal 115 issues and the data is reviewed at 112 for changes that have been recorded over the predetermined time. If changes have been recorded during this period signal 113 is issued and monitoring continues. If no changes in the trend of recorded data for the parameter are detected for the predetermined period a signal 117 will be issued instructing at 118 that all recorded data to be reviewed for data changes from the time the device was activated. If there has been no change of data, signal 119 displays on the interface device 14 and the remote monitor 18 notifying the patient and the clinician of a possible device malfunction. The sensing device 12, the interface device 14 and the patient can be checked to determine source of the problem.

Time periods for transmitting data packets to the interface device 14 are programmed m the sensing device 12 electronics. As mentioned above it is preferred to keep transmission of the packets of digital data to a minimum, particularly at the earlier stage of pregnancy, to preserve battery life. At a later stage or critical period during the pregnancy the period between data packet transmission may be shortened. Likewise the predetermined period for data review at 112 is programmed into the electronics of the interface device 14. Preferably this period can be programmed by the clinician and will be adjusted depending on the parameter being monitored and the stage of the pregnancy. For example, in the early stage of a pregnancy the predetermined period for impedance measurement may be as long as several months. In the later stages, however, change in impedance becomes more critical and the predetermined period may be a few hours.

The data can be displayed by the interface device 14 to provide a read out of any changes or lack thereof from the previously recorded data. Thus, those attending the individual being monitored, or in the case of a human, the patient herself, will receive immediate notification of a problem with the pregnancy or a possible device malfunction. The compared data and data base may be periodically wirelessly transmitted in step 111 through the transmitter 16 to the monitor 18 that may be remotely located for display and monitoring by the clinician. In addition the data can be plotted on a curve, along with the previously recorded data. Alternatively, only a signal indicating a significant change in one or all of the parameters being monitored or a problem with the sensing device 12 or the accumulation device 14 will be transmitted to the monitor 18 to alert the clinician to a potential problem that may require attention.

While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims and their equivalents.

1. A system for monitoring a pregnant mammal, said system comprising:
a. a sensing device for measurement of one or more physiological parameters of the cervix and for generating digital data responsive thereto;
b. interface device for receiving and analyzing said digital data generated by said sensing device and for recording and comparing said data to a database relating to said physiological parameter being measured and for wirelessly transmitting signals relating to said data and said database through a transmitter to a remote unit.

2. The system of claim 1 wherein said interface device is located externally of the body of the individual being monitored

3. The system of claim 1 wherein said sensing device measures at least one of electrical impedance, pH and temperature.

4. The system of claim 1 wherein said sensing device collects said digital data into a packet and transmits said packet at regular intervals.

5. The system of claim 1 wherein said interface device includes software for analyzing said database by comparison thereof to data stored in said interface device.

6. The system of claim 1 wherein said remote unit includes software for analyzing said database by comparison thereof to data stored in said interface device.

7. The system of claim 1 wherein said interface device receives data from said sensor device and compares said received data with baseline data generated from previously received data for an individual being monitored.

8. The system of claim 7 wherein after comparing with said baseline data, said later received data is added to said baseline data.

9. The system of claim 1 wherein said interface device compares said data with a database comprising typical values for the physiological parameters being monitored.

10. The system of claim 1 wherein said remote unit compares said data with a database comprising typical values for the physiological parameters being monitored.

11. The system of claim 1 wherein measurement of the physiological parameters is noninvasive and the individual being monitored is ambulatory.

12. The system of claim 1 wherein said sensing device comprises a biocompatible body defining an outer surface for positioning adjacent said cervix, said body including at least one compartment for a power supply and electronics, at least one parameter sensing electrode disposed on said outer surface for contact with said cervix when said body is positioned adjacent said cervix.

13. The system of claim 1 wherein said interface device comprises a housing containing a receiver and electronics including memory for accumulating data from said sensor device and having software programed to compare received data to base line data stored in said memory.

14. The system of claim 1 wherein said remote unit includes electronics including memory and software to compare received data to base line data stored in said memory.

15. The system of claim 1 wherein said sensing device comprises a nonconductive, flexible annulus defining an inner radial surface, at least one electrode for sensing a physiological parameter of said cervix disposed on said inner radial surface and at least one compartment for a power supply and electronics.

16. The system of claim 1 wherein said sensing device is configured as a constricting pessary.

17. The system of claim 1 wherein said remote unit comprises a server at a central data collection point.

18. The system of claim 1 wherein said remote unit is a monitor in a clinician's office.

19. The system of claim 1 wherein said remote unit is at a hospital where a clinician is located.

20. A method for monitoring a pregnant mammal by the measurement of physiological parameters of the cervix, said method comprising the steps of:
a. sensing at least one physiological parameter of the cervix and generating an electrical signal corresponding to the sensed parameter;
b. converting the electrical signal to digital data;
c. transmitting said digital data to an interface device for analysis;
d. analyzing said digital data by comparing the digital data with a database representative of said physiological parameter being sensed to determine a change in data trend;
e. storing said digital data;
f. continuing said sensing, converting, transmitting and comparing throughout a predetermined period;
g. transmitting an alarm signal in the event a significant change in the trend of said digital data; and
h. transmitting an alarm signal in the event of device malfunction.

21. The method of claim 20, wherein said digital data is compared to a database of typical values for said parameter.

22. The method of claim 20, wherein said digital data is combined with previously received data to generate a database for said parameter being measured.

23. The method of claim 20, wherein said parameter being monitored is selected from the group consisting of impedance, pH and temperature.

24. The method of claim 20, wherein said parameter being monitored is impedance.

25. The method of claim 20, wherein at predetermined time intervals said data is transmitted to a remote unit.

26. The method of claim 25, wherein said remote unit is a central server that collects, analyses and stores data from multiple individuals and issues notification to clinicians responsible for each individual of.

27. The method of 20, wherein transmission of said data is wireless.

28. The process of monitoring a pregnancy for early detection of conditions characteristic of premature birth, which comprises:
a. sensing the physiological parameters of cervical impedance, pH and temperature and generating an electrical signal proportional to the intensity of each parameter;
b. converting the electrical signal to a digital point;
c. transmitting said digital point to an interface device;
d. comparing the digital point with a base line representative each said physiological parameter being sensed;
e. storing said digital point;
f. continuing said sensing, converting, transmitting and comparing with a base of the physiological parameters throughout a predetermined period; and
g. transmitting an alarm signal to a monitor in the event a significant change in a trend of said digital point.

29. A method for monitoring the birth process of a mammal comprising the steps of:
a. determining the condition of the cervix as it relates to the onset of labor and birth by measuring of the physiological parameters of the cervix as a determination of effacement and ripeness;
b. conducting signals generated by the measurement of the physiological parameters to an interface device;
c. processing the data to create a baseline of the physiological parameters;
d. continuously transmitting signals and collecting and processing data therefrom to determine changes in the trend of the data in relation to the baseline;
e. wirelessly transmitting the collected data from the interface device to an transmitter unit; and
f. transmitting the data to a remote unit for display and monitoring by the clinician.
